(19) United States
(12) Patent Application Publication
(10) Pub. No.: US 2009/0288908 A1
(43) Pub. Date: Nov. 26, 2009

(54) Protective-sleeve cartridge and stethoscope incorporating same

(76) Inventors: Jennifer S. Giroux, Cincinnati, OH (US); David A. Daugherty, Columbus, MS (US)

Correspondence Address: DAVID E. PRITCHARD;ATTORNEY AT LAW, 3843 DRAKEWOOD DRIVE, CINCINNATI, OH, 45209, US

(21) Appl. No.: 12/315,256

(22) Filed: Dec. 1, 2008

Related U.S. Application Data
(60) Provisional application No. 61/004,867, filed on Nov. 30, 2007.

(51) Int. Cl. A61B 7/02

(52) U.S. Cl. 181/131

(57) ABSTRACT

A stethoscope protective-sleeve cartridge system of the invention includes a cartridge for mounting to a stethoscope, and for dispensing a protective sleeve into which a head of a stethoscope is placed. The cartridge includes a housing and a plurality of protective sleeves positioned in the housing. The housing defines an interior space and includes an opening through which the protective sleeves are drawn. Each sleeve includes a leading end, a trailing end, an interior, and an opening at a location between the leading and trailing ends. The stethoscope head is passed through the sleeve opening and into a portion of the interior of the sleeve.

### CROSS-REFERENCE TO RELATED APPLICATION

This patent document claims the benefit of the filing date of Provisional Application No. 61/004,867, entitled “Protective-Sleeve Cartridge and Stethoscope Incorporating Same” and filed on Nov. 30, 2007. The entire disclosure of Provisional Application No. 61/004,867 is incorporated into this patent document by reference.

### FIELD OF THE INVENTION

This invention relates to stethoscope covers, and more particularly, to stethoscope covers that cover the head of a stethoscope.

### BACKGROUND OF THE INVENTION

The stethoscope is a clinical, diagnostic instrument used to conduct, or transmit, sounds produced in a patient's body to one or both ears of a clinician. Because of the diagnostic importance of the stethoscope, virtually every clinician has a stethoscope, and uses that same stethoscope throughout the day. For example, a clinician may use their stethoscope during examination of patients in a physician's office, in a hospital clinic, in an emergency room, and while examining hospital inpatients. By using their stethoscope, the clinician is able to transmit respiratory, cardiac, plural, arterial, and other sounds to the ear(s) of the clinician, by applying the head of the stethoscope to the patient's chest, back, abdomen, arms, and other areas. Depending on the particular patient, any one or more of these areas may be secreting bodily fluids contaminated with infectious agents, including, for example, viruses such as the human immunodeficiency virus (HIV), resulting in the contamination of the stethoscope head. And, unless the clinician sterilizes their stethoscope between each patient examination, each subsequent patient's skin could become contaminated with any infectious agents that may have been present on at least the prior patient, and more likely, an accumulation of infectious agents of all of the clinician's preceding patients for the day.

In practice, the vast majority of medical personnel do not clean or sanitize their stethoscopes between examinations of different patients. Accordingly, while helpful and seemingly innocuous, in truth, stethoscopes end up transmitting any of a number of different infectious agents from one patient to another. And depending on the nature of the infectious agents, as well as the health status of the various patients seen by the clinician, an otherwise healthy patient may become sick; and in some cases, a patient may even die as a result of the infectious agents transferred via the contaminated head of the stethoscope.

### SUMMARY OF THE INVENTION

The invention overcomes the drawbacks and limitations described above by providing a stethoscope protective-sleeve cartridge system. To this end, and in accordance with the principles of the invention, in one aspect, the system is directed to a stethoscope and a cartridge mounted to the stethoscope. The cartridge may include a housing and a plurality of protective sleeves positioned in the housing. The housing may define an interior space and may include an opening through which the protective sleeves may be drawn. Each sleeve may include a leading end, a trailing end, an interior, and an opening at a location between the leading and trailing ends; and the sleeve opening may be constructed and arranged to enable the stethoscope head to be passed through the sleeve opening and into a portion of the interior of the sleeve.

In another aspect, the system is directed to a cartridge for mounting to a stethoscope, and for dispensing a protective sleeve into which a head of a stethoscope may be placed. The cartridge may include a housing and a plurality of protective sleeves positioned in the housing. The housing may define an interior space and may include an opening through which the protective sleeves may be drawn. Each sleeve may include a leading end, a trailing end, an interior, and an opening at a location between the leading and trailing ends; and the sleeve opening may be constructed and arranged to enable a stethoscope head to be passed through the sleeve opening and into a portion of the interior of the sleeve.

In a further aspect, each sleeve may be constructed and arranged so that a stethoscope head does not pass through the leading end of the sleeve. For example, the sleeve may include a first side and a second side; and these sides may be affixed to one another at or near the leading end.

In yet another aspect, each sleeve may have a width; and the housing opening through which the sleeves pass may have a length. If desired, the sleeves and the opening may be sized so that the sleeve width is greater than the housing opening length. This aspect can assist in regulating the movement of the sleeves from the housing interior space and through the housing opening.

In a further aspect, the plurality of sleeves may include a length of longitudinally-spaced interconnected sleeves, with one sleeve being separable from an adjacent sleeve. If desired, the sleeves may be in the form of a roll, and the roll may be rotatable relative to the housing. Also, the roll may be connected to a frame. If desired, the frame may be formed so that it is not an integral part of the housing. For example, the frame may be a separate component. If desired, the housing may includes a cover, with the cover defining the housing interior space; and the roll and frame may be positioned in the interior space.

In another aspect, where the plurality of sleeves includes a length of longitudinally-spaced interconnected sleeves, a spacer may be positioned between, and may interconnect, the trailing end of one sleeve and the leading end of an adjacent sleeve. The spacer may include a leading end and a trailing end, with the spacer leading end connected to the trailing end of the one sleeve, and the spacer trailing end connected to the leading end of the adjacent sleeve. The leading end of the spacer may be separable from the trailing end of the one sleeve. And the trailing end of the spacer may be separable from the leading end of the adjacent sleeve.

In yet another aspect, where a spacer is positioned between and interconnects the trailing end of one sleeve and the leading end of an adjacent sleeve, the spacer may include one or more markings. The marking can assist in alerting a user that the adjacent sleeve is approaching the housing opening when the user sees the spacer at the housing opening. If desired, the marking may be printed on an interior surface of the spacer—thereby assisting in inhibiting or preventing the marking from being transferred to an exterior surface of any of the protective sleeves, or onto the skin of a patient that may be contacted with any one of the sleeves.

In a further aspect, each of the sleeves may include a longitudinal axis, and the housing opening may be elongated and generally transverse to the longitudinal axis. In addition, the cartridge may include a fastener operable to releasably grasp a sound tube of a stethoscope.

These and other advantages will become apparent from the accompanying drawings and description of the drawings.

### BRIEF DESCRIPTION OF THE DRAWINGS

The accompanying drawings, which are a part of this specification, illustrate embodiments of the invention. And together with the general description of the invention given above, and the detailed description of the drawings given below, the accompanying drawings explain the principles of the invention. The drawings are schematic, and are not to scale.

FIG. 1 is a perspective view of a protective-sleeve cartridge embodiment mounted to a stethoscope, in accordance with the principles of the invention;

FIG. 2 is a perspective view of the protective-sleeve cartridge of FIG. 1;

FIG. 3 is a perspective view of the roll of protective sleeves of the protective-sleeve cartridge of FIG. 1;

FIG. 4 is a perspective view of the frame and fasteners of the protective-sleeve cartridge of FIG. 1;

FIG. 5 is a perspective view of the protective-sleeve cartridge of FIG. 1, with the cartridge shown in a partially disassembled state;

FIG. 6 is another perspective view of the protective-sleeve cartridge of FIG. 1;

FIG. 7 is a perspective view of a user tearing a spacer off of the roll of protective sleeves of the protective-sleeve cartridge of FIG. 1, with the cartridge mounted to the stethoscope of FIG. 1;

FIG. 8 is a perspective view of the user of FIG. 7 dispensing the next sleeve from the protective-sleeve cartridge of FIG. 1, with the cartridge mounted to the stethoscope of FIG. 1;

FIG. 9 is a perspective view of the user of FIG. 7 placing a portion of the stethoscope of FIG. 1 into the sleeve that the user dispensed in FIG. 8;

FIG. 10 is a perspective view of another embodiment of the protective-sleeve cartridge, in accordance with the principles of the invention, with the cartridge shown in a partially disassembled state; and

FIG. 11 is another perspective view of the embodiment of FIG. 10, with the cartridge shown in a partially disassembled state.

### DETAILED DESCRIPTION OF THE DRAWINGS

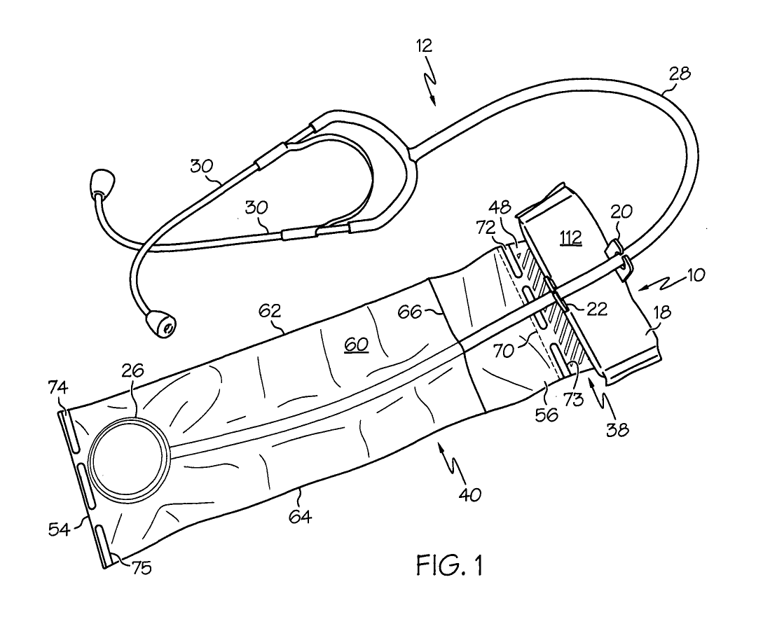

With reference to FIG. 1, one embodiment of the cartridge 10, in accordance with the principles of the invention, is shown mounted to a stethoscope 12. As seen in FIGS. 1-9, and discussed in further detail below, the cartridge 10 includes a housing, a roll 14 of protective sleeves, a frame 16, and a pair of fasteners. The roll 14 is mounted to the frame 16, with the roll and frame positioned inside the housing. In this embodiment 10, the frame 16 is not formed as an integral part of the housing, but rather as a separate component. The particular housing shown is a flexible cover 18. The fasteners extend from the frame 16, and are releasably connected to the stethoscope 12. The particular fasteners illustrated are in the form of generally planar tabs 20, 22. The cover 18 has an elongated overlap opening 24 through which a protective sleeve may be drawn or pulled from the cartridge 10.

The particular stethoscope 12 shown is a binaural stethoscope, although any suitable stethoscope may be used with the cartridge. The stethoscope has a head 26, a sound tube 28 connected to the head 26, and a pair of earpiece tubes 30 connected to the sound tube 28. The cartridge tabs 20, 22 are releasably connected to a portion of the sound tube 28.

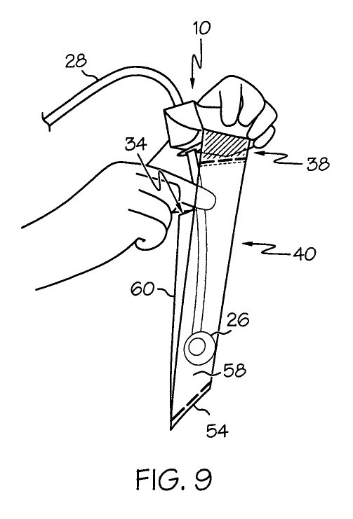

As seen in FIGS. 8 and 9, a sleeve 40 is drawn or pulled from the cartridge 10 by a user (e.g., a clinician); and the stethoscope head 26 and a portion of the sound tube 28 are placed into the sleeve 40 through an access opening 34 formed in the sleeve 40.

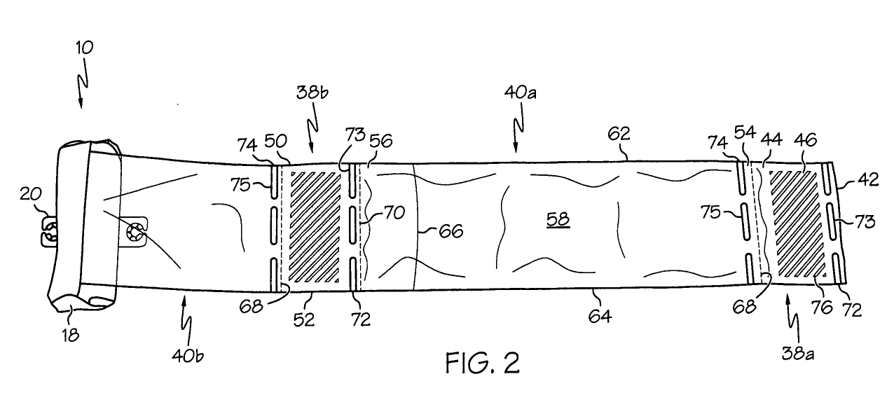

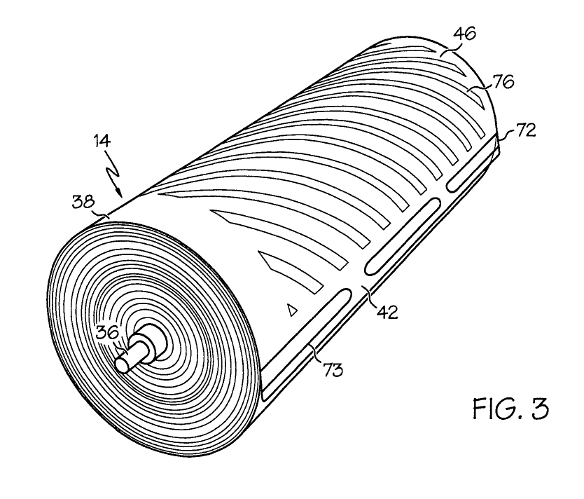

Referencing FIGS. 1-3, the roll 14 includes a length of thin flattened tubular sheeting, with the sheeting being segmented so as to define a series of protective sleeves, as discussed below. An axle 36 is located at the core of the roll 14 (FIG. 3). The sheeting has a leading end (e.g., an outermost end) and a trailing end (e.g. the end at the core of the roll). And the longitudinal axis of the tubular sheeting is perpendicular to the axle 36 and the roll core. The roll 14 of sheeting is segmented into detachably connected spacers (as at 38a, 38b, FIG. 2) and protective sleeves (as at 40a, 40b, FIG. 2)—with each sleeve (as at 40a, FIG. 2) being spaced from the next sleeve (as at 40b, FIG. 2) on the roll by an intervening spacer (as at 38b, FIG. 2), as discussed below. In addition to its leading and trailing ends, the sheeting may be said to include a first side, an oppositely disposed second side, a first longitudinal edge, and a second longitudinal edge. Along the length of the sheeting, the first and second sides are connected to one another at the first and second longitudinal edges. The sheeting may be formed of any suitable material(s), using any appropriate manufacturing technique(s). Advantageously, the sheeting may be made of a non-latex material.

The segmentation of the sheeting into protective sleeves and intervening spacers is accomplished by perforated lines formed in the sheeting, as discussed below. The lines run transverse to the longitudinal axis of the sheeting, and are useful not only in assisting to define the spacers and sleeves, but also in facilitating the separation of a spacer or sleeve from the roll. Each spacer (generally, 38) is identical to the other spacers on the roll; and each sleeve (generally, 40) is identical to the other sleeves on the roll.

With reference to FIGS. 1, 2, 8, and 9, as with the length of sheeting itself, each one of the spacers 38 has a leading end 42, a trailing end 44, a first side 46, an oppositely disposed second side 48 (FIGS. 1 and 2), a first longitudinal edge 50, and a second longitudinal edge 52—with the first and second sides 46, 48 connected to one another at the first and second longitudinal edges 50, 52. In similar fashion, each of the sleeves 40 has a leading end 54, a trailing end 56, a first side 58, an oppositely disposed second side 60, a first longitudinal edge 62, and a second longitudinal edge 64—with the first and second sides 58, 60 connected to one another at the first and second longitudinal edges 62, 64. The second side 60 of each protective sleeve 40 includes an access opening 34 through which the stethoscope head 26 and adjacent portion of the sound tube 28 may be passed into the sleeve interior. As shown, the particular access opening 34 is created by a transverse slit 66 that extends from the first longitudinal edge 62 to the second longitudinal edge 64—with the slit 66 being positioned about four fifths (⅘ths) of the way along the length of the sleeve 40, as measured from the leading end 54 toward the trailing end 56. If desired, the access opening may comprise any suitable shape, and may be positioned at any suitable location along the sleeve.

As best seen in FIG. 2, a perforated line 68 formed in the tubular sheeting between a spacer 38 and an adjacent subsequent sleeve 40 assists in defining the trailing end 44 of the spacer 38 and the leading end 54 of the adjacent subsequent sleeve 40. And a perforated line 70 formed in the tubular sheeting between a sleeve 40 and an adjacent subsequent spacer 38 assists in defining the trailing end 56 of the sleeve 40 and the leading end 42 of the adjacent subsequent spacer 38. In addition, the trailing end of a spacer abuts the leading end of an adjacent subsequent sleeve (until the spacer is detached from the adjacent sleeve, e.g., by tearing the perforated line between the two); and the trailing end of a sleeve abuts the leading end of an adjacent subsequent spacer (until the sleeve is detached from the adjacent spacer, e.g., by tearing the perforated line between the two).

Referencing FIGS. 1-3 and 5-7, the first and second sides 46, 48 of each spacer 38 are connected together at the leading end 42 of the spacer 38 by a transverse line or row 72 of three sealed areas (as at 73)—with each sealed area being spaced from an adjacent sealed area. And in similar fashion, the first and second sides 58, 60 of each protective sleeve 40 are connected together at the leading end 54 of the sleeve 40 by a transverse line or row 74 of three sealed areas (as at 75)—with each sealed area being spaced from an adjacent sealed area. This connection of the sleeve first and second sides 58, 60 is especially beneficial, in that the head 26 of the stethoscope 12 does not pass through the leading end 54 of a sleeve 40 when the head 26 is releasably positioned within the sleeve 40. Any suitable connecting materials or methods (e.g., heat sealing) may be used to connect the first and second sides of each spacer and each sleeve. Also, the spacer and sleeve first and second sides may be connected together at any suitable spots or locations, using any suitable number of sealed areas. If multiple spaced-apart sealed areas are used, openings between the sealed areas can allow air to pass; this aspect can facilitate forming a roll of sheeting. Alternatively, if desired, the sides may be connected (e.g., sealed) via an uninterrupted connection (e.g., sealed line) that extends across the entire width of the sheeting.

As seen in FIGS. 1-3 and 5-7, each spacer 38 includes a number of diagonal stripes (as at 76) that facilitate quick and easy identification of the spacer 38. If desired, the stripes 76 may be red. The stripes 76 are printed on the interior surface of the spacer first side 46. This is particularly advantageous, in that a user does not contact the interior surface of a spacer 38 during normal use and handling of a cartridge 10, and therefore, does not risk transferring printing material (e.g., an ink or the like) to a patient. In addition, the stripe printing material is not transferred to any protective sleeves 40 on the roll 14 of sheeting; and therefore, printing material is not transferred to a patient In other embodiments, if one or more markings or indicia are desired in order to facilitate quick and easy identification of the spacers, any suitable markings or indicia may be used—either at the spacers or elsewhere.

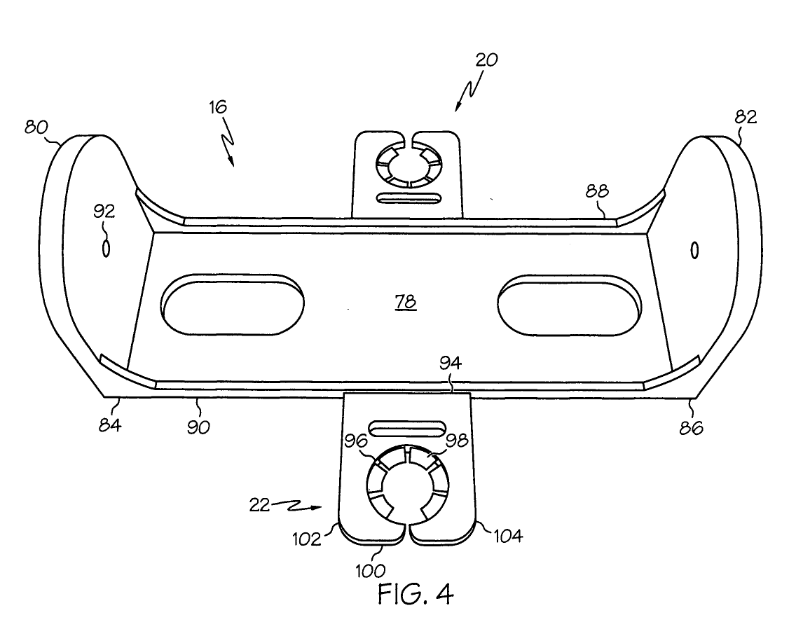

With reference to FIG. 4, the frame 16 includes a wall member 78 and a pair of brackets 80, 82 extending from, and integrally connected to, the wall member. The wall member 78 includes a first end 84, a second end 86, an upper elongated sidewall 88, and a lower elongated sidewall 90. One bracket 80 is located at the first end 84; and the other bracket 82 is located at the second end 86. The brackets 80, 82 are sufficiently spaced from each other so that the roll 14 of protective sleeves (FIG. 3) may be positioned between the brackets, and connected to the brackets via the axle 36. The axle 36 includes a first end section (FIG. 3) and a second end section (not shown) that extend outward beyond the ends of the roll 14. Each bracket 80, 82 includes an opening (as at 92) operable to receive an end section of the axle 36; and each end section is positioned in the corresponding opening. In this fashion, the frame 16 supports the roll 14; and a user may rotate the roll 14 relative to the frame 16, as discussed in further detail below. The frame may be made of any appropriate material(s), employing any suitable manufacturing method(s). If desired, the frame may be made of plastic.

In other embodiments, the roll may be connected to the frame in any suitable manner. For example, each bracket may include any suitable structure or feature for capturing the ends of the axle (e.g., slots, channels, or the like). Alternatively, each bracket may include a post, pin, or the like, that may be inserted into the roll via a corresponding end of the roll—typically at the longitudinal axis core of the roll. If desired, one or more tubes (e.g., rigid or semi-rigid tubule(s)) may be at the roll core; and a post, pin, or similar structure may be inserted into the corresponding tube.

As best seen in FIG. 4, each one of the pair of fasteners is a generally planar tab 20, 22, with tab 22 having the same features as those of tab 20. Each tab 20, 22 includes an inner end 94 integrally connected to the frame wall member 78 via a living hinge (not shown). Tab 20 is connected to the upper elongated sidewall 88, equidistant from the wall member first and second ends 84, 86; and tab 22 is connected to the lower elongated sidewall 90, also equidistant from the wall member first and second ends 84, 86. Each tab 20, 22 further includes a circular rim 96 that defines an opening, and a plurality of semi-flexible finger-like projections (as at 98) that extend radially inward from the circular rim—partway toward the center of the opening. In addition, each tab includes an outer end 100 that is opposite the inner end 94. A slit extends from the outer end 100 to the circular rim 96, and has the effect of creating two arm-like portions 102, 104. Each portion 102, 104 may be flexed above or below the plane of the tab 20, 22, independently of the other portion. In this fashion, a user easily may position the two arm-like portions 102, 104 around the stethoscope sound tube 28, with the finger-like projections forming a friction fit with an outer surface of the sound tube.

The circular rim and projections of each of the tabs may have any suitable dimensions—so as to form a friction fit with one or more differently sized stethoscope sound tubes, while at the same time making it easy for a user to adjust the location of the tabs (and therefore the cartridge) to a desired position along the length of the sound tube. Typically, a user adjusts the position by sliding the tabs along the sound tube. Alternatively, a user may unfasten the tabs from about the sound tube, and reposition the tabs at a different location along the length of the sound tube. Although a particular fastener system has been described, any suitable fastener or combination of fasteners may be used. In addition, the fastener(s) may be made of any appropriate material(s). If they are integrally connected to the frame, then they typically are made of the same material as the frame.

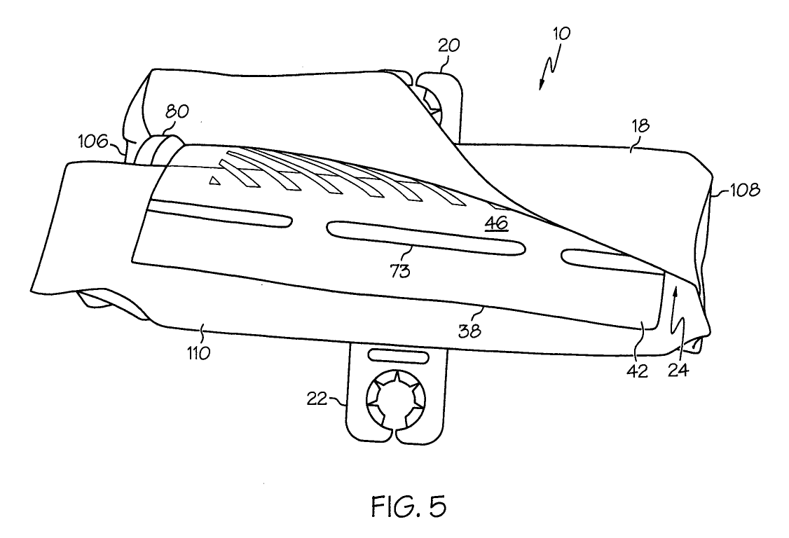

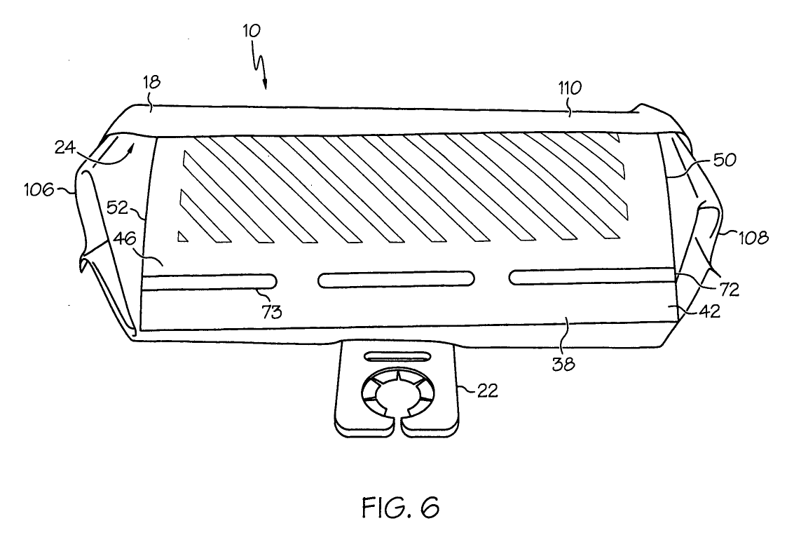

As best seen in FIGS. 5 and 6, the housing is in the form of a flexible cover 18. The cover 18 has a first end 106, a second end 108, and an elongated circumferential sidewall 110 that define an interior space. The roll 14 and frame 16 are positioned within the interior space. The cover 18 further includes an elongated overlap opening 24 that extends generally from the first end 106 to the second end 108, and through which an outermost portion of the length of tubular sheeting extends. In FIGS. 5 and 6, a portion of a spacer 38 is shown extending through the overlap opening 24. In the illustrated embodiment, the cover 18 is made of a highly-flexible drapable plastic sheeting; and the overlap opening 24 is formed by overlapping a portion of the elongated circumferential sidewall sheeting with another portion. In addition, and with reference to FIG. 1, the cover 18 includes a back wall 112 having an upper slot (not shown) and a lower slot (not shown). The tab 20 extends outward through the upper slot; and the tab 22 extends outward through the lower slot. In other embodiments, the housing may have any suitable shape and construction, and may be made of any appropriate material(s).

When a user is ready to attach a cartridge (e.g., a new cartridge) to a stethoscope, it is helpful to select a “working” location for the cartridge, along the length of the stethoscope sound tube. With reference to FIGS. 1 and 9, one example of a working location for the cartridge 10 is a location that enables the head 26 of the stethoscope 12 to be at or near the leading end 54 of the protective sleeve 40 when the head 26 and a portion of the sound tube 28 are positioned in the interior of the protective sleeve 40. If the outermost spacer and sleeve are not already positioned outside the interior space of the cover (e.g., exposed), the user may draw them from the interior space by holding the cover, and pulling on an exposed leading portion of the spacer at the elongated overlap opening. In this fashion, the roll of sheeting will rotate relative to the frame and cover; and the user will draw the spacer and sleeve from the interior space. The user should stop pulling on the sheeting when the sleeve trailing end and adjacent spacer leading end are visible at the overlap opening. The user then may mount the cartridge on the stethoscope by positioning the tabs about the sound tube. In addition, the user may remove the exposed spacer and sleeve from the next spacer, e.g., by tearing the perforated line at the trailing end of the exposed sleeve. Advantageously, a user may keep the cartridge 10 connected to the stethoscope 12 until all of the cartridge sleeves 40 have been used.

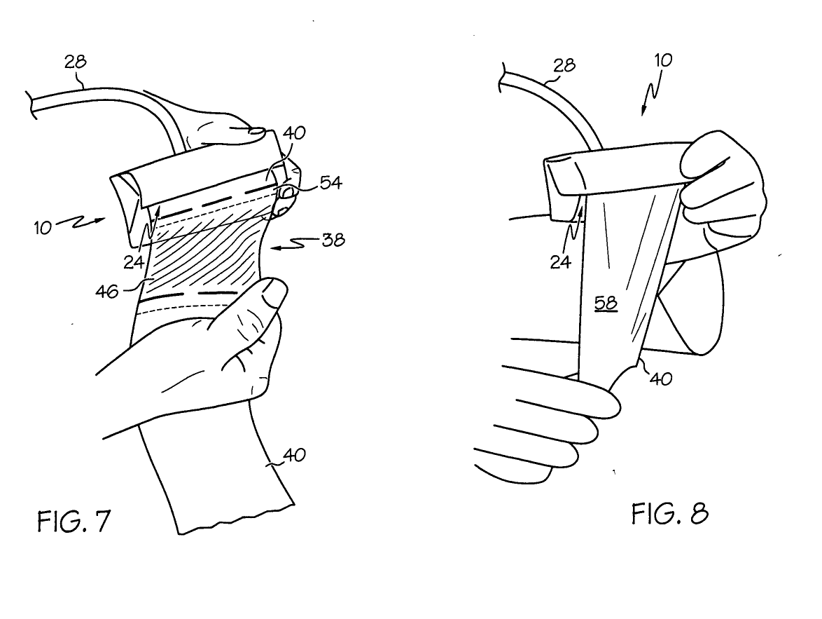

Referencing FIGS. 7-9, prior to examining a patient with the stethoscope, the user may remove the next spacer 38 from the leading end 54 of the next sleeve 40—keeping the leading end 54 of the next sleeve 40 near the elongated overlap opening 24. If the outermost exposed spacer or sleeve described in the preceding paragraph has not yet been separated from the cartridge roll 14, the user may do so at this point. The user then puts on a pair of gloves, and draws this next sleeve 40 from the interior space by pulling on an exposed leading portion of the sleeve 40 (FIG. 8). The user stops drawing out the sleeve 40 when the subsequent spacer (38, FIG. 9) begins to appear at the overlap opening 24. At this point, the user inserts the stethoscope head 26 and an adjacent portion of the sound tube 28 through the access opening 34 formed in the second side 60 of the sleeve 40, and into an interior space defined by the sleeve. Advantageously, the user positions the head 26 within the sleeve 40 such that the head is near the leading end 54 of the interior space of the sleeve. With the protective sleeve 40 in place, the user now may examine the patient with the stethoscope 12.

After examining the patient, the user may hold the cartridge 10, tear the perforated line at the trailing end of the now-used sleeve, and discard the used sleeve. Prior to examining the next patient with the stethoscope, the user may remove the next spacer from the leading end of the next sleeve, put on a pair of gloves, and repeat the quick simple steps described above.

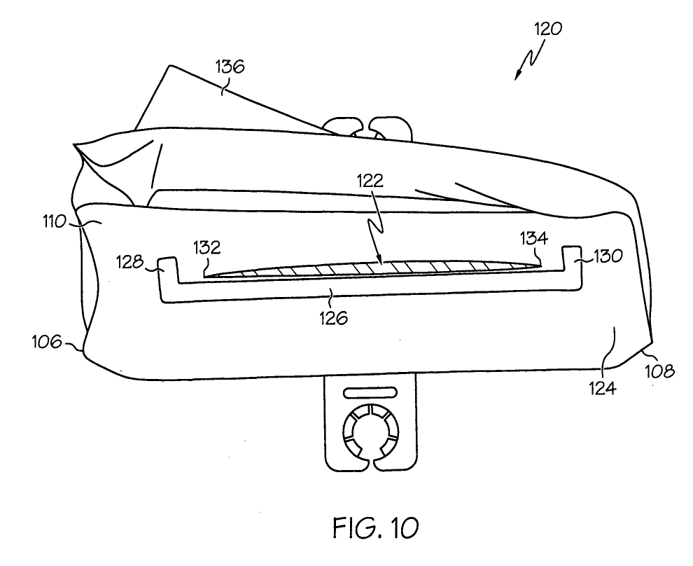

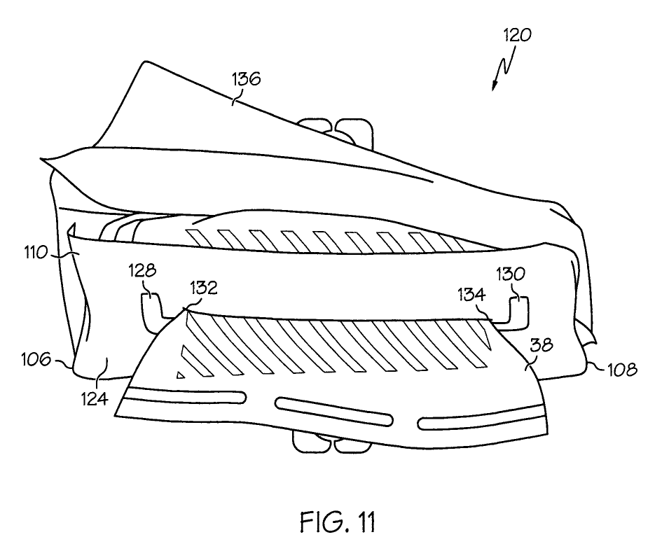

With reference to FIGS. 10 and 11, another cartridge embodiment 120 is shown. This cartridge 120 is substantially similar to the cartridge 10 described above. Unlike cartridge 10 however, cartridge 120 has a horizontal elongated dispensing opening 122 (FIG. 10) formed in a front wall portion 124 of the flexible cover sidewall 110, through which the tubular sheeting (spacers 38 and protective sleeves 40) is dispensed. Because cartridge 120 is substantially similar to cartridge 10, like reference numbers are used to identify like components, elements, and features; and in general, a description of those like aspects is not repeated here. As shown, the dispensing opening 122 is created by forming a horizontal elongated slit in the front wall portion 124. The front wall portion 124 further has a line (formed by heat or the like) adjacent the dispensing opening 122. The line includes a horizontal portion 126 (FIG. 10) that runs just below the opening 122, and an upwardly extending leg 128, 130 at each end of the horizontal portion 126. The leg 128 is positioned to the left of the left end 132 of the opening 122; and the leg 130 is positioned to the right of the right end 134 of the opening. In FIG. 10, the cartridge 120 is shown without the tubular sheeting extending through the dispensing opening 122—in order to show the opening 122 and horizontal portion 126 of the adjacent line more clearly. In FIG. 11, the cartridge 120 is shown with a portion of a spacer 38 extending through the dispensing opening.

The length of the opening 122 is shorter than the length of the cartridge 120—the length of the opening 122 being about three fifths (⅗ths) of the distance from the cover first end 106 to the cover second end 108, with the opening being centrally positioned between the ends 106, 108. The length of the opening 122 also is shorter than the width of the tubular sheeting on the roll 14—the length being about three fourths (¾ths) of the width of the tubular sheeting. In other embodiments, the elongated dispensing opening may have any suitable relative length. The relatively shorter length of opening 122 can assist in regulating the movement of tubular sheeting from the interior space of the cover 18 to the outside.

In its fully assembled state (not shown), the cartridge 120 has an outer overlap opening (not shown) substantially similar to the elongated overlap opening 24 of cartridge 10 (see, e.g., FIGS. 5 and 6). This outer overlap opening is formed by having a portion 136 of the flexible cover sidewall 110 extend over most of the front wall portion 124, including over the elongated dispensing opening 122—thereby “hiding” the opening 122 from view; however, the opening 122 remains fully functional. In use, a leading portion of the tubular sheeting typically extends between these overlapped portions 124, 136, and out from the cover 18, where it may be readily grasped by a user.

While the present invention has been illustrated by a description of various embodiments, and while the illustrative embodiments have been described in considerable detail, it is not the intention of the inventors to restrict or in any way limit the invention to such detail. Additional advantages and modifications will readily appear to those skilled in the art. The invention, in its broader aspects, is therefore not limited to the specific details, representative apparatus and methods, and illustrative examples shown and described. Accordingly, departures may be made from such details without departing from the spirit or scope of the inventors' general concept.

What is claimed is:

1. A stethoscope protective-sleeve cartridge system, comprising:
a stethoscope including a head; and
a cartridge mounted to the stethoscope, the cartridge comprising:
a housing defining an interior space and including an opening through which a protective sleeve may be drawn; and
a plurality of protective sleeves positioned in the housing and dispensable through the housing opening, each sleeve including a leading end, a trailing end, an interior, and an opening at a location between the leading and trailing ends, the sleeve opening constructed and arranged to enable the stethoscope head to be passed through the sleeve opening and into a portion of the interior of the sleeve.

2. A stethoscope protective-sleeve cartridge system, comprising:
a cartridge for mounting to a stethoscope, and for dispensing a protective sleeve into which a head of a stethoscope may be placed, the cartridge comprising:
a housing defining an interior space and including an opening through which a protective sleeve may be drawn; and
a plurality of protective sleeves positioned in the housing and dispensable through the housing opening, each sleeve including a leading end, a trailing end, an interior, and an opening at a location between the leading and trailing ends, the sleeve opening constructed and arranged to enable a stethoscope head to be passed through the sleeve opening and into a portion of the interior of the sleeve.

3. The system of claim 2 wherein each sleeve is constructed and arranged so that a stethoscope head may not pass through the leading end.

4. The system of claim 2 wherein each sleeve has a width, and wherein the housing opening has a length, the sleeve width being greater than the housing opening length.

5. The system of claim 2 wherein the plurality of sleeves comprises a length of longitudinally-spaced interconnected sleeves, with one sleeve being separable from an adjacent sleeve.

6. The system of claim 5 wherein the plurality of sleeves is in the form of a roll, and the roll is rotatable relative to the housing.

7. The system of claim 6 wherein the roll is connected to a frame.

8. The system of claim 7 wherein the frame is not formed as an integral part of the housing.

9. The system of claim 8 wherein the housing includes a cover, the cover defining the housing interior space, the roll and frame positioned in the interior space.

10. The system of claim 5 including a spacer positioned between and interconnecting the trailing end of the one sleeve and the leading end of the adjacent sleeve.

11. The system of claim 10 wherein the spacer includes a leading end and a trailing end, the spacer leading end connected to the trailing end of the one sleeve, and the spacer trailing end connected to the leading end of the adjacent sleeve.

12. The system of claim 11 wherein the leading end of the spacer is separable from the trailing end of the one sleeve.

13. The system of claim 11 wherein the trailing end of the spacer is separable from the leading end of the adjacent sleeve.

14. The system of claim 10 wherein the spacer includes a marking, the marking operable to assist in alerting a user that the adjacent sleeve is approaching the housing opening when the user sees the spacer at the housing opening.

15. The system of claim 14 wherein each sleeve includes an exterior surface, wherein the spacer includes an interior surface, and wherein the marking is printed on the spacer interior surface, thereby assisting in inhibiting or preventing the marking from being transferred to the sleeve exterior surface or onto the skin of a patient that may be contacted with any one of the plurality of protective sleeves.

16. The system of claim 2 wherein each of the plurality of sleeves includes a longitudinal axis, and the housing opening is elongated and generally transverse to the longitudinal axis.

17. The system of claim 2 wherein the cartridge includes a fastener, the fastener operable to releasably grasp a sound tube of a stethoscope.
